(19) United States
(12) Patent Application Publication
(10) Pub. No.: US 2012/0028321 A1
(43) Pub. Date: Feb. 2, 2012

(54) High Solids Fermentation for Synthesis of Polyhydroxyalkanoates From Gas Substrates

(76) Inventors: Craig S. Criddle, Redwood City, CA (US); Wei-Min Wu, Mountain View, CA (US); Gary D. Hopkins, Milpitas, CA (US); Eric R. Sundstrom, Menlo Park, CA (US)

(21) Appl. No.: 12/901,343

(22) Filed: Oct. 8, 2010

Related U.S. Application Data
(60) Provisional application No. 61/278,682, filed on Oct. 8, 2009.

(51) Int. Cl. C12P 7/42

(52) U.S. Cl. 435/146

(57) ABSTRACT

Production of polyhydroxyalkanoates (PHAs) is performed by delivering substrates such as methane in gas phase during a high solids fermentation. Microorganisms are grown under balanced conditions, then gas phase substrates are delivered under unbalanced conditions to produce PHA granules inside the cells. The cells containing these granules are lysed and the bioplastic powder recovered. The balanced phase growth may occur in submerged liquid cultures or attached as biofilms to a surface.

### CROSS-REFERENCE TO RELATED APPLICATIONS

This application claims priority from U.S. Provisional Patent Application 61/278,682 filed Oct. 8, 2009, which is incorporated herein by reference.

### FIELD OF THE INVENTION

The present invention relates generally to methods and devices for the production of bioplastics and resins. More specifically, it relates to the biosynthesis of polyhydroxyalkanoate (PHA) bioplastics and resins using bacteria that can use gaseous alkanes and alkenes for PHA synthesis.

### BACKGROUND OF THE INVENTION

Conventional microbial production of bioplastic polyesters is performed using fermenting liquid-phase bioreactors. The production typically involves cycling through two growth phases. First, pure or mixed cultures of bacteria are grown under balanced growth conditions, i.e., with sufficient carbon feedstock and nutrients for cell division. Next, the biomass is grown under unbalanced conditions, i.e., with sufficient feedstock (typically a sugar) but lacking one or more essential nutrients (e.g., N or P). During this period of unbalanced growth, many bacteria produce polyhydroxyalkanoate (PHA) granules, e.g., polyhydroxybutyrate (PHB) and/or polyhydroxyvalerate (PHV). In conventional techniques for such biosynthesis of PHA, both phases of production involve submerged (liquid-phase) growth, i.e., all of the feedstock is supplied through the aqueous phase. Cells containing PHA granules can be harvested by filtration or centrifugation and lysed. Dissolution of the PHA into a separate liquid phase (organic solvents, supercritical CO₂, etc.) may then be used to remove the residual non-PHA cell debris. Alternatively, dissolution of the non-PHA phase (e.g., acid-base treatment) can be used to isolate undissolved PHA. There are, however, limitations associated with method for biosynthesis of PHA. It would be desirable to increase growth rates so that bioplastics can be more rapidly produced, readily switch feedstocks to modify the composition of PHA produced, decrease water requirements for PHA production, and increase the energy efficiency of PHA biosynthesis.

### SUMMARY OF THE INVENTION

Gaseous carbon substrates offer several advantages for growth of PHA-producing microorganisms, but their low aqueous solubility limits culture density, specific growth rate, and PHA production rate. Maintenance of high substrate levels in the water phase leads to inefficient use of substrates and high demand for energy.

The present inventors have recognized that terrestrial plants solve a similar problem with minimal energy investment by extracting carbon and oxygen from the gas phase. Motivated by this insight, the inventors have developed a similar strategy for bioreactor design to enable an efficient, low-energy means of growing and harvesting bioplastic-rich biomass.

In contrast with prior methods in which the substrates used to produce polyhydroxyalkanoates (PHAs) are delivered through liquid phase fermentations, the present invention provides methods in which gas substrates (methane, propane, butane, etc.) are delivered in gas phase to microbial biofilms in a high solids fermentation. The gas phase delivery of these substrates addresses the mass transfer problem associated with use of poorly soluble gaseous substrates and provides a simple and practical way to produce bioplastics.

This technique significantly increases the rate of mass transfer to cells, enabling more rapid production of the bioplastic, enables delivery of diverse gas phase substrates for co-polymer production, and improves opportunities for PHA extraction. Diffusion of a substrate through the gas phase is 10,000 times faster than diffusion through liquid water, and does not require the high energy inputs necessary for mass transfer through liquid.

According to preferred embodiments of the invention, the unbalanced growth conditions are established in a high solids fermentation, thus avoiding the mass transfer limitations associated with delivery of poorly soluble substrates, such as methane and oxygen into a water phase. In one specific implementation, type II methanotrophs are grown under the appropriate selection conditions (i.e., balanced growth), separated from the liquid phase, then transferred to a chamber. In the chamber, gas phase substrates (methane, propane, butane, oxygen) are delivered under unbalanced conditions, i.e., where nitrogen and other nutrients required for balanced growth are not present, and the gas phase substrates are consumed to produce bioplastic granules inside the cells. The cells containing these granules are then lysed and the bioplastic powder recovered.

Production of PHA is thus a two-step process: (1) balanced growth in which cells accumulate, and (2) unbalanced growth in which cells expand as PHA accumulates within the cells. In a preferred embodiment, submerged growth is carried out for step (1) and solid-phase fermentation for step (2), but both steps can also be carried out in a high solids fermentation

Embodiments of the invention provide methods for producing polyhydroxyalkanoate (PHA). The methods include providing a chamber containing microorganisms in the form of moist biofilms, delivering gas phase carbon substrates to the chamber for production of PHA in a high-solids fermentation, and extracting PHA from the microorganisms. The production of PHA takes place during unbalanced growth conditions in the chamber. In some cases, the method may include delivering nutrients to the chamber for balanced growth of the microorganisms in the chamber. The nutrients may be delivered in liquid phase. The moist biofilms may have various forms such as films covering moist particles in the chamber or films covering moist membrane sheets in the chamber. The nutrients may be delivered to the biofilms through the membrane sheets.

The methods of the present invention provide a simple and economic technique to produce diverse polyhydroxyalkanoate (PHA) bioplastics and resins from low-cost gaseous substrates, such as biogas methane derived from organic wastes. The methods and devices of the present invention provide low-cost production of bioplastics that can replace conventional synthetic plastics and resins derived from petrochemical feedstocks.

### BRIEF DESCRIPTION OF THE DRAWINGS

FIG. 1 is a schematic diagram of a configuration for producing PHA through a high solids fermentation according to an embodiment of the invention.

FIG. 2 is a schematic diagram of a configuration for producing PHA through a high solids fermentation according to another embodiment of the invention.

FIG. 3 is a schematic diagram of membrane sheets used in a chamber for producing PHA through a high solids fermentation according to an embodiment of the invention.

### DETAILED DESCRIPTION

Embodiments of the present invention provide for improvements in the production of polyhydroxyalkanoates (PHAs) by communities of microorganisms in bioreactors. During unbalanced growth, a nutrient such as N or P limits growth and biopolymer accumulates inside the cells. In contrast with conventional methods, in embodiments of the present invention the unbalanced growth occurs in a high solids phase in which the carbon feedstock provided as a gas. In the context of the present invention, “high solids” refers to the use of biomass within a chamber that is filled predominately with gas rather than liquid fluids, where the biomass is composed of at least 20% solid phase biomass by volume in an attached or immobilized state within the chamber.

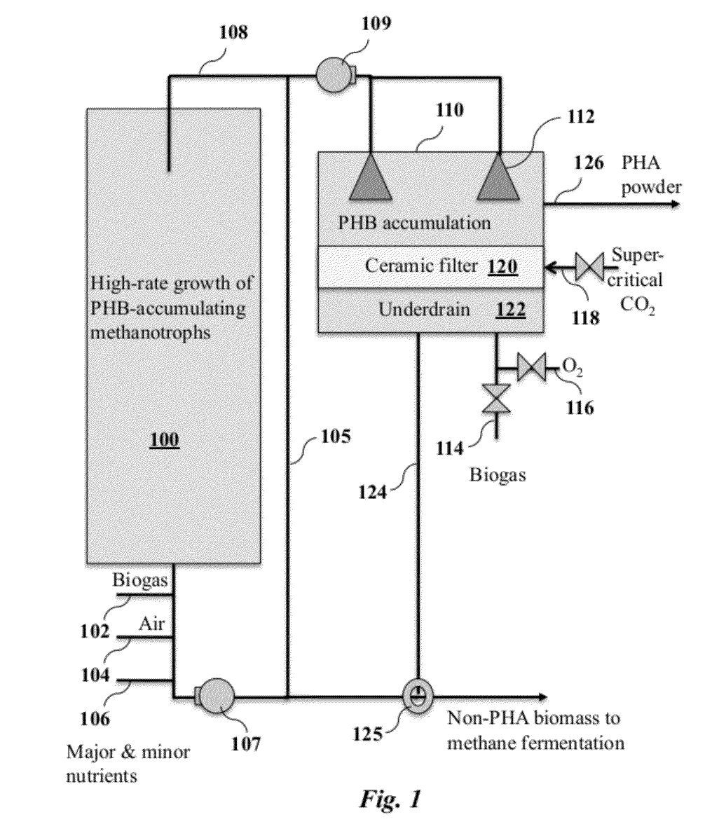

FIG. 1 is a schematic illustration of one possible bioreactor configuration that may be used to achieve high solids polyhydroxyalkanoate (PHA) production from gaseous alkanes according to an embodiment of the invention. A bioreactor vessel 100 contains a culture of bacteria selected for PHA accumulation. The volatile hydrocarbon substrate, oxygen, and nutrients (major and minor) are provided to the vessel through inlets 102, 104, 106, respectively, to establish and maintain balanced growth conditions in the vessel. Accordingly, the bacteria multiply at a high rate. In this embodiment, the balanced growth in vessel 100 takes place in the liquid phase, i.e., the feedstock and nutrients are mixed with the bacteria in aqueous solution.

Outlet 108 carries bacteria from vessel 100, through pump 109, and into a high solids fermentation chamber 110. Some bacteria may be recirculated through line 105 and pump 107 back into reactor 100. In the high solids fermentation chamber 110, the microorganisms may take the form of moist biofilms on a support 112 within the chamber 110. Gas phase carbon substrates are then delivered to the chamber, taken up by the moist biofilms, resulting in the production of PHA in a high-solids fermentation. For example, biogas and oxygen in gas phase enter the chamber 110 through inlets 114 and 116, respectively. The lack of nutrients in chamber 110 creates unbalanced growth conditions and results in the accumulation of PHA within the bacteria. Extraction of the PHA from the microorganisms is performed by delivering supercritical carbon dioxide or another suitable solvent to the chamber 110 through inlet 118. A ceramic filter 120 in the chamber 110 catches the PHA granules and allows cellular debris to pass down in an underdrain 122 in the chamber where it is collected and then exits the chamber via outlet 124. The cellular debris biomass flows through valve 125. The biomass exiting the system may be used, for example, in methane fermentation. The PHA granules are removed from the chamber 110 via outlet 126 and used for the subsequent production of bioplastic materials.

Solid-state fermentation within chamber 110 involves the use of a support 112 for the growth of the biofilm as well as means for delivery of substrates to the biofilms through the gas phase. The support 112 for biofilm growth may be made of non-biodegradable materials, such as fabrics, ceramics, plastics, and porous membranes, or biodegradable supports, such as wheat straw or cellulosic materials.

Gas phase substrates may be include a variety of substances, including 1) carbon sources such as biogas, natural gas (CH₄), propane, butane, ethane, ethylene, CO, CO₂, etc., 2) nitrogen sources such as N₂ and NH₃, 3) electron acceptors such as O₂ and N₂O, and 4) electron donors such as H₂.

In some embodiments, the process may also involve a phase of growing the biofilms in the chamber using liquid phase delivery of nutrients and trace elements. Such liquid phase delivery can be achieved using a percolate spray, mist, or periodic immersion of biomass in a bath or rotating drum, sprinkled permeate, aerosols, or passage of biofilm biomass on a disk or drum that rotates through a bath. Again, a variety of substrates can be delivered providing considerable flexibility in the incubation conditions. Substrates that may be delivered through the liquid phase may include 1) carbon sources such as volatile fatty acids, sugars and other soluble organics, 2) nitrogen sources such as nitrate and ammonium, 3) electron acceptors such as dissolved oxygen and nitrate, 4) trace nutrients such as phosphorus, sulfur, and essential cations, and 5) electron donors such as formate.

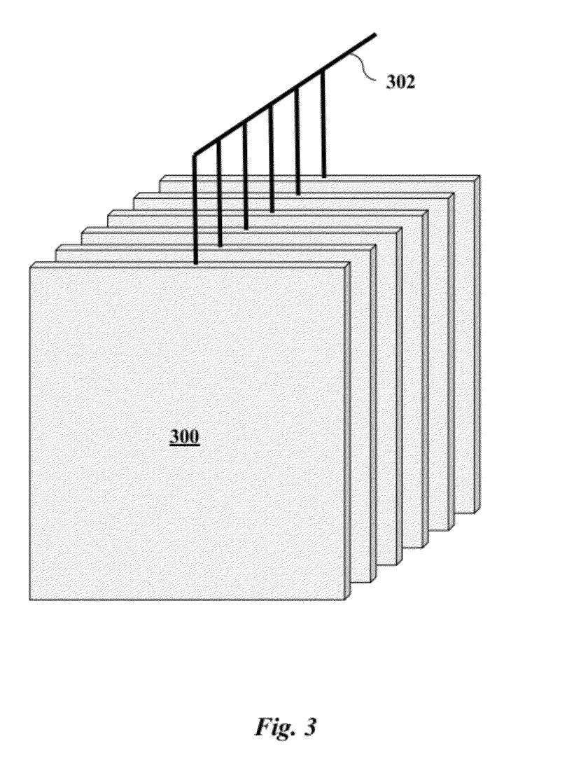

In another embodiment, biofilms are attached to the exterior of gas- and water-permeable membrane curtains 300, as shown in FIG. 3. The curtains 300 may be suspended within a humid chamber 110 as part of a system as described in relation to FIG. 1. During a balanced growth phase, water and nutrients are delivered from supply line 302 into the interior of the membranes 300 where they permeate into biofilms that are attached to the outer surface of the membranes. The biofilms are exposed to the gas phase within the chamber. Upon switching to unbalanced growth conditions (i.e., no longer providing the biofilm with nutrients via the membrane), biopolymer will accumulate in biofilm cells. An outer shell of polymer-rich biofilm is then stripped off, leaving behind a thin biofilm for re-growth in the next cycle of PHA production. Various techniques may be used to strip the outer layer of the biofilm, including mechanical means to assist in the stripping process. Membrane curtains may be composed of materials such as polytetrafluoroethylene mesh, silk, rayon, polyester or cotton. A support rack and liquid distribution system may be used in a multi-curtain reactor.

In preferred embodiments, methane biogas is used as a low-cost carbon source for PHA synthesis. Type II methanotrophs are used to produce polyhydroxybutyrate (PHB), a useful bioplastic, when grown with methane under unbalanced growth conditions. Cells grown by conventional means under balanced conditions are harvested then subjected to unbalanced growth conditions in a solid-state fermentation. As described above in relation to FIG. 1, this may be accomplished by transferring cells to a chamber where gas phase substrates (e.g., methane, propane, butane, oxygen) are delivered. Under such conditions, nitrogen and other nutrients required for balanced growth are not present, and gas phase substrates are used to produce bioplastic granules inside the cells. Cells containing these granules can then be lysed and the bioplastic powder recovered.

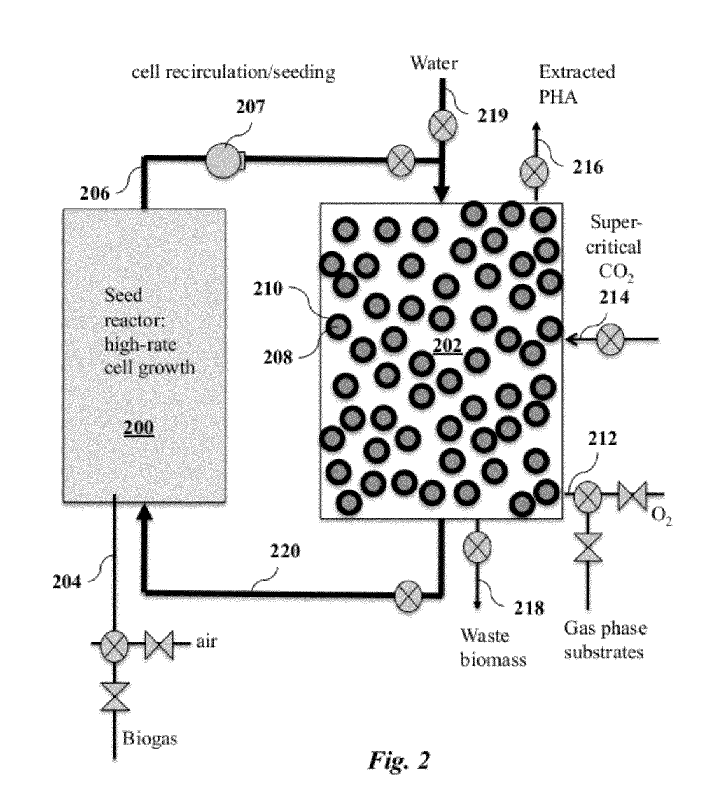

FIG. 2 illustrates another possible configuration according to an embodiment of the invention. Methanotrophs are continuously grown at a high rate in a suspended growth or attached-growth bioreactor 200 that is designed to select for rapid growth of Type II methanotrophs. This selection is achieved by control of dissolved oxygen, pH, and nitrogen source. The Type II methanotrophs are periodically harvested and sprayed onto a filter bed within a sealed chamber 202 flushed with biogas methane and oxygen. PHB accumulation ensues. The PHB-rich cells are then lysed and the PHB extracted using supercritical CO₂. Non-PHB cell material is flushed to an anaerobic bioreactor for fermentation back into biogas.

The configuration shown in FIG. 2 may be used to perform high solids fermentation for PHB production using absorbed water and nutrient and a seed reactor. In seed reactor 200, type II methanotrophic cells are grown at high rate with biogas (50:50 CH₄:CO₂), air and other nutrients for balanced growth, provided to the reactor through inlet 204. Seed reactor 200 may be operated in the manner of conventional liquid phase bioreactors. Cells are drawn from bioreactor 200 through outlet 206 and pump 207 and injected into a solid-state fermentation chamber 202 containing moist absorbent materials to which the cells adhere. For example, the moist absorbent materials may be moist particles 208, and the cells may form a biofilm 210 on the surface of the particles. Oxygen and gas phase substrates, such as methane, are introduced into the solid-state fermentation chamber 202 through inlet 212. As the gas circulates in the chamber, passing between the particles and coming into contact with the biofilm 210 layers, the cells grow under unbalanced growth conditions, accumulating PHB. The accumulated PHB is obtained by supercritical CO₂ extraction, in which supercritical CO₂ is introduced into the chamber through inlet 214, PHB granules are removed through outlet 216, and non-PHB waste biomass is flushed from the chamber through outlet 218 after which it may be subsequently digested in an anaerobic digester to produce new biogas. Inlet 219 is provided for the delivery of water to the chamber. Recirculation line 220 allows waste biomass from chamber 202 to be fed back into reactor 200.

Several variations of the above techniques are possible. In one variation, balanced growth may be performed in chamber 202 in a separate phase from unbalanced growth by introducing liquid with nutrients into the chamber, e.g., through inlet 219. In another variation, cells may be harvested from liquid culture in the seed reactor 200 via centrifugation, passage through a bag filter, membrane separation, or dissolved air flotation. In another variation, the cells can be incubated in the presence of different gas phase substrates to create different useful polyhydroxyalkanoates. More specifically, by changing gas phase substrates (from biogas methane to propane or butane, for example) or by modifying the composition of the percolate/bath water composition to include a soluble carbon source, such as propionate or butyrate, during the high solids fermentations, co-polymers, such as PHBV or different PHA molecules such as polyhydroxyhexamoate (PHHx) are produced with different properties than PHB, extending the range of possible applications for the bioplastics that are produced.

In some variants of the above embodiments, submerged growth of cells during balanced growth can be achieved using dispersed-growth suspensions or attached growth, in which cells grow upon a carrier, such as activated carbon particles. Dispersed cells can be harvested by centrifugation, passage through a filter, membrane separation, or dissolved air flotation then subject to high solids fermentation for PHA accumulation. Attached growth cells can be detached, concentrated then subject to unbalanced growth conditions in a high solids fermentation.

The embodiments of the present invention have several important advantages over conventional submerged-only fermentations. First, embodiments of the invention have lower energy expense for delivery of a wide range of substrates during the PHA accumulation phase. In liquid reactors, delivery of substrates is a major operational expense because energy is required for mixing of the liquid. Gas phase diffusion coefficients are 10,000 times higher than liquid phase diffusion coefficients. This translates into much faster rates of mass transfer and less energy inputs for delivery of substrates to the biomass. Second, embodiments of the present invention require smaller reactor volumes than comparable liquid phase reactors. Less space is required for PHA production, less water is required, and PHA-enriched biomass can potentially be extracted within the same tank, minimizing solids and water handling and capital costs. Third, embodiments of the invention allow easy delivery of mixed substrates. Gases can be easily removed from an incubation chamber, and new gases introduced. This enables modification of the fermentations to enable production of PHA block co-polymers or mixed PHA polymers that have improved properties for specific applications.

1. A method for producing polyhydroxyalkanoate (PHA) comprising:
providing a chamber containing microorganisms in the form of moist biofilms;
delivering gas phase carbon substrates to the chamber for production of PHA in a high-solids fermentation; and
extracting PHA from the microorganisms.

2. The method of claim 1 wherein the production of PHA takes place during unbalanced growth conditions in the chamber.

3. The method of claim 1 further comprising delivering nutrients to the chamber for balanced growth of the microorganisms in the chamber.

4. The method of claim 3 wherein the delivery of nutrients to the chamber comprises delivering the nutrients in liquid phase.

5. The method of claim 1 wherein the moist biofilms have the form of films covering moist particles in the chamber.

6. The method of claim 1 wherein the moist biofilms have the form of films covering moist membrane sheets in the chamber.

7. The method of claim 6 further comprising delivering nutrients to the biofilms through the membrane sheets.
